(19) United States
(12) Patent Application Publication
(10) Pub. No.: US 2010/0240718 A1
(43) Pub. Date: Sep. 23, 2010

(54) NOVEL HETEROARYL-SUBSTITUTED ACETONE DERIVATIVE, SUITABLE FOR INHIBITING PHOSPHOLIPASE A2

(75) Inventors: Matthias Lehr, Havixbeck (DE); Stefanie Bovens, Munster (DE)

Correspondence Address: SCHMEISER, OLSEN & WATTS, 22 CENTURY HILL DRIVE, SUITE 302, LATHAM, NY, 12110, US

(73) Assignee: WESTFALISCHE WILHELMS UNIVERSITÄT MÜNSTER, Muenster (DE)

(21) Appl. No.: 12/678,870

(22) PCT Filed: Sep. 19, 2008
(86) PCT No.: PCT/EP2008/062552

§ 371 Date: Mar. 18, 2010

(30) Foreign Application Priority Data
Sep. 21, 2007 (DE) 10 2007 045 476.9

(51) Int. Cl. A61K 31/4245; C07D 209/12; A61K 31/404; A61P 25/28; A61P 11/06; A61P 29/00; A61P 27/14; C07D 413/04

(52) U.S. Cl. 514/364; 548/493; 514/419; 548/131

(57) ABSTRACT

The present invention relates to novel heteroaryl-substituted acetone derivatives inhibiting the enzyme phospholipase A2, and pharmaceutical agents comprising said compounds.

### BACKGROUND OF THE INVENTION

The present invention relates to novel heteroaryl-substituted acetone derivatives that inhibit the enzyme phospholipase A₂. These compounds are suitable as medicine for prevention and treatment of diseases, which are caused or contributed to by an increase in activity of this enzyme, such as inflammations, pain, fever, allergies, asthma, psoriasis, and endotoxic shock.

By the term “phospholipase A₂” is meant the large and diverse group of enzymes that cleave the phospholipids at the sn-2 position producing free fatty acids and lysophospholipids.

If arachidonic acid is one of the released fatty acids, this can be metabolized to prostaglandins and thromboxanes over the cyclooxygenase pathway and to leukotrienes and other hydrolyzed fatty acids over the lipoxygenase pathways. The prostaglandins play an important role in the development of pain and fever and inflammatory reactions. Leukotrienes are important mediators in inflammation processes and in anaphylactic and allergic processes. The lysophospholipids formed by phospholipase A₂ have cytotoxic properties. Lysophosphatidylserine leads to the release of a histamine involved with allergic processes. In addition, Lysophosphatidylcholine will metabolize to platelet activating factor (PAF), which is also an important mediator for example in inflammation processes.

An excessive stimulation of the phospholipase A₂ can therefore lead to a series of acute and chronic illnesses.

### BRIEF SUMMARY OF THE INVENTION

In the prior art, inhibitors of the cytosolic phospholipase A₂ are known. For example, the paper WO 2004/069797, which is referenced in its entirety, disclosed heteroaryl-substituted acetone derivatives, which inhibit the enzyme phospholipase A₂.

There is a need for novel inhibitors of phospholipase A₂, in particular of cytosolic phospholipase A₂.

It was therefore necessary to provide novel compounds that inhibit the enzyme phospholipase A₂.

This need is met through the compounds of the general formula (I) as specified below:

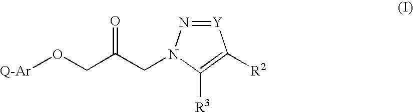

wherein
Q represents R¹, OR¹, SR¹, SOR¹, SO₂R¹, NR⁹R¹ or a straight-chained C₁₋₃₁ alkyl or C₂₋₃₁ alkenyl or alkynyl residue, which may be interrupted by 1 or 2 residues, independently chosen from O, S, SO, SO₂, NR⁹, and aryl, which can be substituted with 1 or 2 substituents R⁴, and which can be substituted with 1 to 4 C₁₋₆ alkyl residues and/or 1 or 2 aryl residues, whereby the aryl residues can be substituted with 1 or 2 substituents R⁴;
Ar represents an aryl residue, which can be substituted with 1 or 2 substituents R⁴;
X represents N or CR⁵;
R¹ represents H or an aryl residue, which can be substituted with 1 or 2 substituents R⁴;

### R² and R³

a) Independently represent H, C₁₋₆ alkyl, C₂₋₆ alkenyl, C₂₋₆ alkynyl, or R⁷—W, or
b) together with the carbon atoms to which they are bound, represent a 5- or 6-membered aromatic or heteroaromatic ring, which can be substituted with 1 or 2 substituents R⁴;
   R⁴ represents C₁₋₆ alkyl, halogen, CF₃, CN, NO₂, OR⁹, S(O)_{O}R⁹, COR⁹, COOR⁹,
   CONR⁹R¹⁰, SO₃R⁹, SO₂NR⁹R¹⁰, tetrazolyl or R⁷—W;
   R⁵ represents H or R⁴;
   R⁷ represents C₁₋₆ alkyl, C₂₋₆ alkenyl, or C₂₋₆ alkynyl;
   R⁹ represents H, C₁₋₆ alkyl, or aryl;
   R¹⁰ represents H or C₂₋₆ alkyl;
   W represents COOH, SO₃H, or tetrazolyl; and
   o represents 0, 1, or 2;
   and/or their enantiomers, diastereomers, as well as their pharmaceutically acceptable salts and/or esters, whereby
Y represents CR¹²,
wherein
R¹² is chosen from the group comprising 3-methyl-1,2,4-oxadiazol-5-yl and/or COR¹³,
R¹³ is chosen from the group comprising CF₃, E and/or D-E;
E is chosen from the group comprising COOH, COOR¹⁴, CONR¹⁴R¹⁵, SO₃R¹⁴, and/or SO₂NR¹⁴R¹⁵;
D is chosen from the group comprising C₁₋₁₀ alkyl, C₂₋₁₀ alkenyl, or C₂₋₁₀ alkynyl, aryl, T-aryl, T-aryl-G and/or aryl-G;
T,G are chosen identically or independently of each other from the group comprising C₁₋₁₀ alkyl, C₂₋₁₀ alkenyl, and/or C₂₋₁₀ alkynyl;
R¹⁴, R¹⁵ are chosen identically or independently from the group comprising H, C₁₋₆ alkyl, and/or aryl.

### DETAILED DESCRIPTION OF THE INVENTION

It was unexpectedly found that the novel heteroaryl-substituted acetone derivatives that inhibit the enzyme phospholipase A₂ are able to provide an improved water solubility compared to well-known compounds and/or a good or even improved inhibitory effect.

Also advantageously applicable are pharmaceutically acceptable addition salts of the inventive compounds.

The pharmaceutically acceptable salts can be base-addition salts. These include salts of the compounds with inorganic bases, like alkali hydroxides, alkali earth hydroxides, or with organic bases like mono-, di-, or tri-ethanolamine.

Also advantageously applicable are acid-addition salts, in particular with inorganic acids such as hydrochloric acid, sulfuric acid, or phosphoric acid, or with suitable organic carboxylic or sulfonic acids, or with amino acids.

Usable pharmaceutically acceptable esters of compounds comprise, in particular, physiologically-easily hydrolyzable esters, such as alkyl, pivaloyloxymethyl, acetoxymethyl, phthalidyl, indanyl, and methoxymethylene esters.

Unless otherwise defined, the term “alkyl” comprises straight-chained, branched, or cyclical alkyl groups, such as methyl, ethyl, propyl, butyl, pentyl, neopentyl, undecyl, dodecyl, pentadecyl, hexadecyl, heptadecyl, octadecyl, cyclohexyl, etc.

The term “alkenyl” comprises straight-chained, branched, or cyclical alkenyl groups, such as ethenyl, propenyl, butenyl, decenyl, heptadecenyl, cyclohexenyl, etc.

The term “alkynyl” comprises straight-chained, branched alkynyl groups, such as ethynyl, propynyl, butynyl, decynyl, heptadecynyl, etc.

The term “aryl” comprises phenyl, naphthyl, biphenyl, as well as 5- or 6-membered heterocyclic rings, containing 1 to 3 atoms chosen from O, N, or S and optionally annulated using a benzene ring. Preferred are phenyl and indolyl, especially phenyl.

The term “halogen” comprises a fluorine, chlorine, bromine, or iodine atom, whereby fluorine or chlorine atoms in particular are preferred.

If residues such as R⁴, R⁷, R⁹, and/or R¹⁰ occur several times in a compound, these can each be selected independently from each other.

The straight-chained C₁₋₃₁ alkyl, or C₂₋₃₁ alkenyl, or alkynyl residue, denoted by Q in formula (I), can be interrupted with 1 or 2 residues, independently chosen from O, S, SO, SO₂, NR⁹, and aryl. In the present case, by “interrupted” is meant that in addition to the carbon atoms of its chain, the residue may contain such a residue both at any site within the chain and at the end of the chain, that is, between the carbon chain and Ar. The existing substituents, which might additionally be present, where appropriate, in the form of 1 to 4 C₁₋₆ alkyl residues and/or 1 or 2 aryl residues may be bound to any carbon atom of the chain.

In advantageous embodiments of the inventive compound,
R¹² represents CO—(CH₂)ᵣ—COOR¹⁴,
wherein
r is 1, 2, 3, 4, or 5.

Especially preferably, r is 2, 3, or 4. Preferably, R¹⁴ is chosen from the group comprising H, methyl, and/or ethyl.

In further advantageous embodiments of the inventive compound
D represents —(CH₂)ₛ-aryl-(CH₂)ₜ—
wherein
s, t is identically or independently of each other 0, 1, 2, 3, 4, or 5.

Preferably, s is 0 or 1 and/or t is 0, 1, or 2. Especially preferably, D is chosen from the group comprising —CH₂-aryl-(CH₂)₂— and/or —CH₂-aryl-.

Preferably, R¹² is furthermore chosen from the group comprising CO-aryl-COOH, CO—CH₂-aryl-COOH and/or CO—CH₂-aryl-(CH₂)₂—COOH.

In preferred embodiments of the inventive compounds, Q represents C₅-C₁₂ alkyl, preferably C₇-C₁₀ alkyl. Exceptionally preferably, Q represents C₈-alkyl.

In further preferred embodiments of the inventive compound, Q represents OR¹, wherein R¹ represents an aryl residue, which can be substituted with a substituent R⁴, whereby R⁴ preferably represents CF₃. The substituent R⁴ is preferably bonded in para position.

In the inventive compounds of the formula (I), Ar represents an aryl residue and preferably an aryl residue as previously defined. Especially preferably, Ar represents a phenyl residue, which preferably binds the adjacent groups Q and O together in para position.

Preferably, R² and R³, together with the carbon atoms to which they are bound, form a 6-membered aromatic ring, preferably a benzene ring. This 6-membered aromatic ring can be substituted with 1 or 2 substituents R⁴, whereby 1 substituent R⁴ is preferable. Preferably, the substituent R⁴ is chosen out of the group comprising COOH and/or CONH₂. Especially preferably, R⁴ is COOH.

In preferred embodiments the inventive compounds exhibit a structure according to the general formula (V) as stated hereafter.

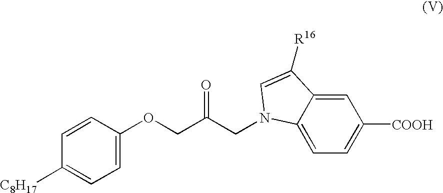

wherein:
R¹⁶ is chosen from the group including —CO(CH₂)₂COOH, —CO(CH₂)₃COOH, —CO(CH₂)₄COOH and/or 3-methyl-1,2,4-oxadiazol-5-yl.

An especially preferred embodiment of the inventive compounds exhibits the following formula (1) and/or their pharmaceutically acceptable esters or salts:

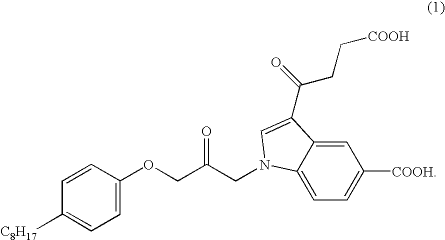

In the context of the present invention, compounds numbered with Arabic numerals differ from compounds numbered with Roman numerals, that is, each deals with different compounds.

Furthermore, an especially preferred embodiment of the inventive compounds exhibits the following formula (2) and/or their pharmaceutically acceptable esters or salts:

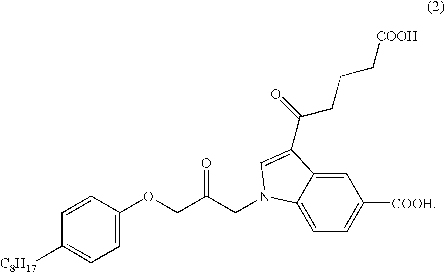

Another especially preferred embodiment of the inventive compounds exhibits the following formula (3) and/or their pharmaceutically acceptable esters or salts:

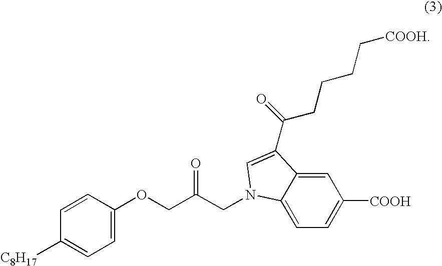

Still another especially preferred embodiment of the inventive compounds exhibits the following formula (4) and/or their pharmaceutically acceptable esters or salts:

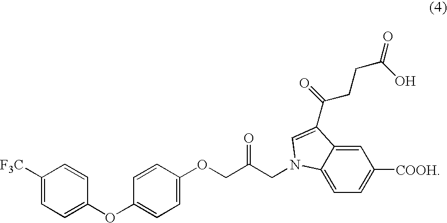

It was unexpectedly found that the inventive compounds exhibit, at least somewhat, good solubility in water. In particular, the compounds according to formulas (1) to (4) feature good water solubility.

In preferred embodiments the solubility of the compounds in aqueous phosphate buffer (pH 7.4) ranges from 10 μg/ml to 500 μg/ml, preferably from 150 μg/ml to 450 μg/ml, especially preferably from 190 μg/ml to 410 μg/ml.

The water solubility of the compounds was determined by administering aqueous phosphate buffer (pH 7.4) to each respective compound, and the dissolved amount was determined after shaking and centrifugation, as is described in example 12.

Poor water solubility of medicines presents a major obstacle for adequate bioavailability. Adequate bioavailability of a medicine is an essential requirement for its effectiveness. Good water solubility can therefore be especially advantageous when a substance is used as medicine.

In particular, improved water solubility can provide the advantage that the inventive compounds, for example after being orally administered, can dissolve in the gastrointestinal tract to an increased extent.

A special advantage of using the inventive compounds also arises from the fact that in order to attain adequate bioavailability for medicines not easily dissolved in water, solvents such as dimethyl sulfoxide (DMSO) or other surfactants facilitating solubility must be added to the medicines before oral administration. Since these solubility facilitators show cytotoxic effects, a considerable improvement in compatibility can be provided by sufficiently water-soluble medicines for which the use of solubility facilitators is not necessary.

A preferred embodiment of the inventive compounds exhibits the following formula (5) and/or their pharmaceutically acceptable esters or salts:

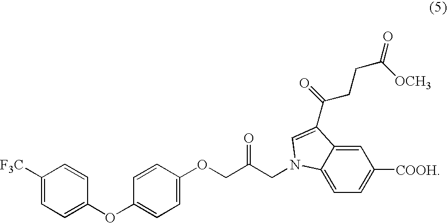

Another preferred embodiment of the inventive compounds exhibits the following formula (6) and/or their pharmaceutically acceptable esters or salts:

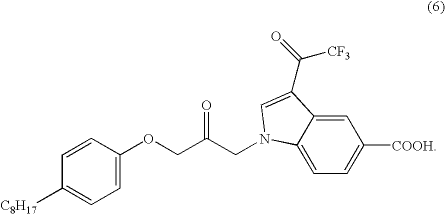

Another preferred embodiment of the inventive compounds exhibits the following formula (7) and/or their pharmaceutically acceptable esters or salts:

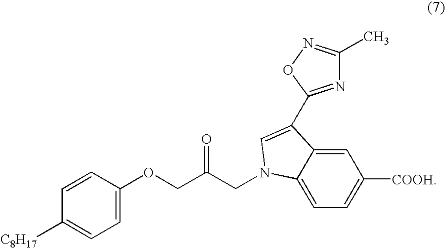

A further preferred embodiment of the inventive compounds exhibits the following formula (8) and/or their pharmaceutically acceptable esters or salts:

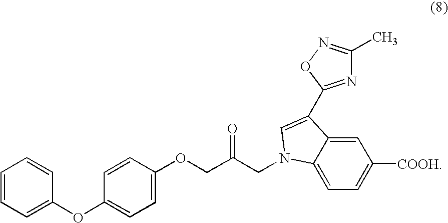

A particular advantage of the inventive compounds arises hereby from the fact that they can provide good inhibition of the phospholipase A₂. In particular, the compounds according to the formulas (6), (7), and (8) can provide especially good inhibition.

A preferred embodiment of the inventive compounds exhibits the following formula (12) and/or their pharmaceutically acceptable esters or salts:

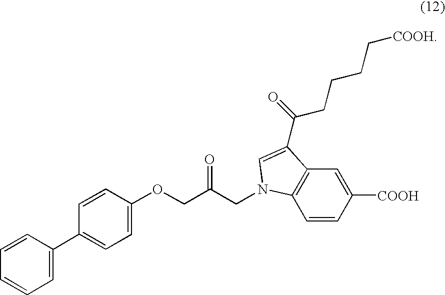

A further preferred embodiment of the inventive compounds exhibits the following formula (13) and/or their pharmaceutically acceptable esters or salts:

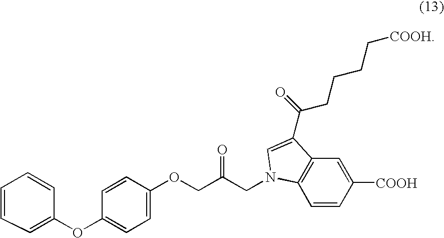

Another preferred embodiment of the inventive compounds exhibits the following formula (14) and/or their pharmaceutically acceptable esters or salts:

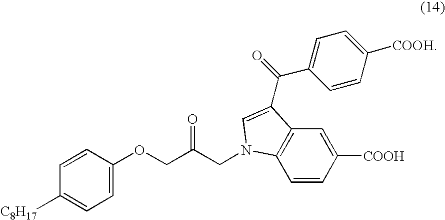

The effectiveness of the inventive compounds is determinable by referring to the inhibition of the cytosolic phospholipase A₂. For this purpose, cytosolic phospholipase A₂ that had been isolated from human thrombocytes was used. To measure enzyme activity, or enzyme inhibition, the arachidonic acid was determined that had been released by the enzyme from 1-stearoyl-2-arachidonoyl-sn-glycero-3-phosphocholine, for example, by reversed phase-HPLC with UV-detection near 200 nm after purification by way of solid-phase extraction.

The inhibition of the enzyme by an inventive compound results from the proportion of the amounts of arachidonic acid formed in the presence, or absence, of the compound.

In preferred embodiments the inventive compounds exhibit IC₅₀ values for the inhibition of cytosolic phospholipase A₂ ranging from 0.001 μM to 0.5 μM, especially preferably ranging from 0.002 μM to 0.3 μM, most preferably ranging from 0.02 μM to 0.25 μM.

The IC₅₀ value of the compounds for the inhibition of cytosolic phospholipase A₂ corresponds to the concentration of the compounds that is necessary to reduce the activity of the enzyme by 50%.

The IC₅₀ values were calculated from the values of cytosolic phospholipase A₂ inhibition obtained from different concentrations with the help of the Probit model (see Hartke, Mutschler, DAB 9 Kommentar Band 1 S. 733-734, Wissenschaftliche Verlagsgesellschaft Stuttgart 1978).

The inventive compounds advantageously show an effective inhibition of phospholipase A₂.

In preferred embodiments the inventive compounds show effective phospholipase A₂ inhibition and good water solubility. In particular, the compounds according to formulas (1) to (5) and (8), particularly according to formulas (1) to (4), feature effective phospholipase A₂ inhibition and good water solubility.

For example, the compounds are useable as medicine for the prevention and treatment of diseases that are caused or contributed to by products or reaction products of this enzyme, for example for the treatment of illnesses in the category of rheumatic diseases and for prevention and treatment of illnesses induced by allergies.

The inventive compounds can therefore be effective analgesics, antiphlogistics, antipyretics, antiallergics, and broncholytic agents and are useable for thrombosis prophylaxis, and for prophylaxis of anaphylactic shock as well as for treating dermatological diseases such as psoriasis, urticaria, acute and chronic rashes of allergic and non-allergic origin.

The inventive compounds can advantageously exhibit, in particular, an anti-inflammatory effect. The inventive compounds can therefore be particularly effective antiphlogistics.

Therefore, the present invention also relates to pharmaceutical agents or medicines, comprising compounds of the general formula (I), particularly compounds according to formulas (1) to (8) and (12) to (14), and/or their enantiomers, diastereomers, as well as their pharmaceutically acceptable salts or esters.

The compounds according to the formula (I), in particular the compounds according to formulas (1) to (8) and (12) to (14) are suited for production of a pharmaceutical agent or medicine for prevention or treatment of illnesses that are caused by or contributed to by an increased activity of phospholipase A₂, preferably of cytosolic phospholipase A₂.

The invention concerns, therefore, in particular the application of the inventive compounds of the general formula (I), especially the compounds according to formulas (1) to (8) and (12) to (14) and/or their enantiomers, diastereomers, as well as their pharmaceutically acceptable salts and/or esters for the production of a pharmaceutical agent or medicine for prophylactic and/or therapeutic treatment of illnesses that are caused by or contributed to by an increased activity of phospholipase A₂.

The term “prophylactic treatment”, in the context of the present invention, especially means that the inventive compounds can be administered prophylactically before symptoms of an illness appear or the danger of an illness exists. In particular, “prophylactic treatment” refers to preventative medication.

Illnesses that are caused by or contributed to by an increased activity of phospholipase A₂ are preferably chosen from the group comprising inflammations, pain, fever, allergies, asthma, psoriasis, cerebral ischemia, Alzheimer's disease, chronic skin diseases, damage to the skin by UV rays, rheumatic illnesses, thrombosis, anaphylactic shock, urticaria, acute and chronic rashes and/or endotoxic shock.

The invention concerns, therefore, in particular the application of the inventive compounds of the general formula (I), particularly compounds according to (1) to (8) and (12) to (14) and/or their enantiomers, diastereomers, as well as their pharmaceutically acceptable salts and/or esters for the production of a pharmaceutical agent or medicine for prophylactic and/or therapeutic treatment of illnesses chosen from the group comprising inflammations, pain, fever, allergies, asthma, psoriasis, cerebral ischemia, Alzheimer's disease, chronic skin diseases, damage to the skin by UV rays, rheumatic illnesses, thrombosis, anaphylactic shock, urticaria, acute and chronic rashes and/or endotoxic shock.

The inventive compounds are especially suitable for treatment of inflammations, preferably for treatment of inflammatory skin diseases or inflammatory diseases of the gastro-intestinal tract.

Preferred inflammatory skin diseases, also called dermatitis, are chosen from the group comprising contact dermatitis, atopic dermatitis, dermatitis solaris, psoriasis, urticaria, acute and chronic rashes of allergic or non-allergic origin, and/or eczema.

In the context of the present invention, the term “eczema” refers to a skin disease that manifests itself as a non-contagious inflammatory reaction of the skin. In the context of the present invention, the term “rash” refers to an inflammatory skin change that often affects a larger area of the skin.

Preferable eczemas are in particular chosen from the group comprising allergic contact eczema, chronic hand eczema, atopic eczema, and/or seborrheic eczema. Preferable rashes of allergic origin are, for example, rashes resulting from an allergy to medication.

Preferable inflammatory diseases of the gastro-intestinal tract are in particular inflammatory bowel disease such as Crohn's disease and/or ulcerative colitis.

The inventive compounds can be administered as individual therapeutic agents or as mixtures with other therapeutic agents. They can be administered alone, preferably in the form of a pharmaceutical agent, that is, as mixtures of the agents with suitable pharmaceutical carriers and/or diluent.

The compounds or pharmaceutical agents can be administered orally, parenterally, transmucosally, pulmonarily, enterally, by inhalation, rectally, or topically, especially dermally, transdermally, bucally, or sublingually.

The type of the pharmaceutical agent and of the pharmaceutical carrier or diluent depends on the desired mode of administration. Oral agents may, for example, be available as tablets or capsules, also as slow-release (retard) form, and can contain conventional excipients, such as binders (e.g. syrup acacia, gelatin, sorbitol, tragacanth, or polyvinylpyrrolidone), fillers (e.g. lactose, sugar, corn starch, calcium phosphate, soribtol, or gylcine), lubricants, (e.g. magnesium stearate, talcum, polyethylene glycol, or silicon dioxide), disintegrating agents (e.g. starch), or wetting agents (e.g. sodium lauryl sulfate). Oral liquid preparations may be available as aqueous or oil suspensions, solutions, emulsions, syrups, elixirs, or sprays, etc., or they may be available as dry powder for reconstitution with water of another suitable carrier. These types of liquid preparations can contain conventional additives, such as suspending agents, flavoring additives, diluents, or emulsifiers. For parenteral administration, conventional pharmaceutical carriers can be employed with solutions or suspensions. For administration by inhalation, the compounds may, for example, be present in a powdery, aqueous, or semi-aqueous solution, which can be used as an aerosol. Preparations for topical application can be available as pharmaceutically acceptable powders, lotions, salves, creams, gels, or as therapeutic systems, which contain therapeutically effective amounts of the inventive compound.

Preferable are, for example, transdermal therapeutic systems such as plaster containing the active agent.

It is especially preferred when the preparation is formed in formulations suited to topical administration. Especially preferred are liquid or semi-liquid preparations, in particular aqueous administration forms for topical application, for example, in the form of solutions or suspensions that can be applied as drops. Lotions, salves, gels, or creams are also preferred.

The necessary dosage depends, for example, on the form of the pharmaceutical agent used, on the mode of use, on the severity of symptoms, and on the type of subject, particularly human or animal, that is being treated. The treatment is usually begun with a dose that is below the optimal dose. Thereafter, the dose is increased until the optimal effect for the given situation is reached.

Preferably, the inventive compounds are administered in concentrations that achieve effective outcomes without having dangerous or disadvantageous effects.

For example, for a topical administration, the agent can be formulated ranging from ≧0.001 wt.-% to ≦10 wt.-%, preferably ranging from ≧0.1 wt.-% to ≦5 wt.-%, especially preferably ranging from ≧1 wt.-% to ≦2 wt.-%, in terms of the total weight of the formulation.

For a topical administration, preferred dosages of the inventive compounds range from ≧0.001 mg/cm² to ≦2 mg/cm² where area refers to application area, particularly skin area, preferably ranging from ≧0.01 mg/cm² to ≦1 mg/cm², especially preferably ranging from ≧0.1 mg/cm² to ≦0.5 mg/cm².

The inventive compounds can be administered in a single dose or in multiple doses.

The inventive compounds according to the general formula (I) are preferably producible according to method disclosed in the publication WO 2004/069797, which is referenced in its entirety, with the exception that for production of the inventive compounds, respectively suitable educts are used.

The inventive compounds according to the general formula (I) are especially preferably producible by converting a compound according to the following general formula (IV)

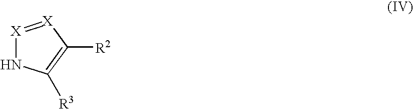

with epichlorohydrin to a compound according to the following general formula (VI)

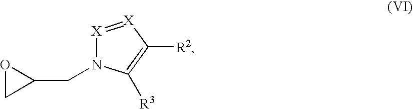

and further, by converting the compound from formula (VI) with a compound according to the following general formula (VII)

Q-Ar—OH (VII)

to a compound according to the following general formula (VIII)

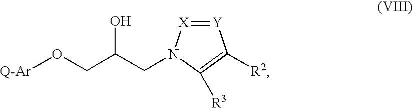

and by oxidizing the compound (VIII) to ketone, whereby the above description is referenced for groups X, Y, Q, Ar, R², and R³.

In the case of the inventive compounds of formula (I), which contain COOH groups, the COOH groups can be protected as ester, preferably as methyl, tert-butyl, benzyl, and allyl. The removal of the ester protecting groups occurs after the oxidation to ketone with known methods. Optionally, the ketone group is hereby protected as acetal.

Examples that help illustrate the present invention are given below.

All batches were carried out in a nitrogen protective gas atmosphere. For column chromatography purification, silica gel 60 (Merck, Darmstadt, Germany) was used, with particle size 63-200 μm or 15-40 μm (=flash chromatography).

### Example 1

### Production of the compound according to formula (1), 3-(3-carboxypropanoyl)-1-[3-(4-octylphenoxy)-2-oxopropyl]indole-5-carboxylic acid

### A. Production of methyl-3-(3-methoxycarbonylpropanoyl)indole-5-carboxylate

5.1 ml (8.39 mmol) n-Butyllithium (1.6 M in hexane) were slowly added by drops to a mixture of 4.2 ml (9.24 mmol) zinc chloride diethyl ether complex solution (2.2 M in methylene chloride) and 20 ml pure methylene chloride under nitrogen at 0° C. over a septum. After complete addition, the reaction mixture was stirred for 1 hour at room temperature (20° C.-23° C.). Then 1.50 g (8.56 mmol) of methylindole-5-carboxylate dissolved in 10 ml pure methylene chloride was added thereto. The mixture was initially stirred for 1 hour at room temperature, then at 0° C. carefully mixed with 2.2 ml (18.1 mmol) succinic acid monomethyl ester chloride and again stirred for 1 hour at room temperature. Finally, aluminum chloride was added and the mixture was again stirred for 1 hour at room temperature. The preparation was poured into half-saturated aqueous NaCl solution and was exhaustively extracted with an ethyl acetate/tetrahydrofuran mixture (7:3). After washing the combined organic phases with saturated NaCl solution and drying over natrium sulfate the solution was filtered and the solvent removed. The product was isolated as solid material from ethyl acetate through recrystallization.

### B. Production of benzyl-3(3-benzyloxycarbonylpropanoyl)indole-5-carboxylate

789 mg (2.73 mmol) methyl-3-(3-methoxycarbonylpropanoyl)indole-5-carboxylate from Step A, were dissolved into 18 ml (0.17 mol) of pure benzyl alcohol and mixed with 0.4 ml (1.91 mmol) titanium(IV) ethoxide. The reaction mixture was heated for 27 hours to 100° C. After cooling to room temperature, the benzyl alcohol was removed by distillation (15 mbar, 95° C.). The distillation residue was absorbed into 20 ml ethyl acetate and the product not dissolved was sucked off over a glass funnel filter. The filtrate was evaporated and the residue was recrystallized from ethyl acetate. The combined solid materials were dried in a vacuum dry box at 40° C.

### C. Production of benzyl-3(3-benzyloxycarbonyl propanoyl)-1-oxiranylmethylindole-5-carboxylate

400 mg (0.91 mmol) benzyl-3(3-benzyloxycarbonylpropanoyl)indole-5-carboxylate from step B were mixed with 102 mg (1.81 mmol) powdered 88% potassium hydroxide and 29 mg (0.09 mmol) tetra-butylammonium bromide. After addition of 4.0 ml (51.1 mmol) epichlorohydrin, the mixture was stirred at room temperature until complete conversion of the educt. Then the preparation was applied directly onto a silica gel column Elution with ethyl acetate/hexane (step gradient: 1:9-1:1) delivered the product as oil.

### D. Production of benzyl-3(3-benzyloxycarbonylpropanoyl)-1-[2-hydroxy-3-(4-octylphenoxy)propyl]indole-5-carboxylate

A mixture of 210 mg (0.42 mmol) benzyl-3(3-benzyloxycarbonylpropanoyl)-1-oxiranylmethylindole-5-carboxylate from step C, 10 mg (0.08 mmol) 4-dimethylaminopyridine, and 87 mg (0.42 mmol) 4-octylphenol was stirred under nitrogen for 20 minutes at 120° C. The preparation was dissolved in a little toluene and the solution applied directly to a silica gel column. The product was obtained as oil after elution with ethyl acetate/hexane (step gradient: 3:7-1:1).

### E. Production of benzyl-3(3-benzyloxycarbonylpropanoyl)-1-[3-(4-octylphenoxy)-2-oxopropyl]indole-5-carboxylate

A solution of 0.6 ml (6.35 mmol) acetic anhydride in 5 ml pure dimethylsulfoxide (DMSO) was stirred for 10 minutes at room temperature. Then this solution was added drop-wise to a solution of 117 mg (0.17 mmol) benzyl-3(3-benzyloxycarbonylpropanoyl)-1-[2-hydroxy-3-(4-octylphenoxy)propyl]indole-5-carboxylate from step D in 5 ml of pure DMSO. After 18 hours of stirring at room temperature, the reaction solution was poured into a mixture of 5% aqueous sodium hydrogen carbonate and saturated aqueous NaCl solution (1:1) and stirred for 10 minutes. After exhaustive extraction with diethyl ether, the combined organic phases were washed three times with saturated aqueous NaCl-solution. After drying over natrium sulfate, the solution was filtered and the solvent removed. After column chromatographic purification on silica gel (ethyl acetate/hexane 3:7), the product was obtained as oil.

### F. Production of 3-(3-Carboxypropanoyl)-1-[3-(4-octylphenoxy)-2-oxopropyl]indole-5-carboxylic acid

10 mg of 10% palladium on activated carbon were added to a solution of 35 mg (0.05) benzyl-3(3-benzyloxycarbonylpropanoyl)-1-[3-(4-octylphenoxy)-2-oxopropyl]indole-5-carboxylate from step E in tetrahydrofuran. After rinsing the apparatus with nitrogen, a hydrogenating balloon filled with hydrogen was attached and hydrogenated for 15 minutes while being stirred at room temperature. Thereafter, it was filtered over absorbent cotton and the solvent was removed. The raw product was purified on silica gel (ethyl acetate/hexane/formic acid 3:7:0.5). The product was dissolved in acetonitrile. After adding water, the acetonitrile was initially removed by distillation and then the water was removed by freeze drying, whereby the product according to formula (1) remained as solid material.

### Example 2

### Production of the compound according to the formula (2), 3-(4-carboxybutanoyl)-1-[3-(4-octylphenoxy)-2-oxopropyl]indole-5-carboxylic acid

### A. Production of methyl-3-(4-methoxycarbonylbutanoyl)indole-5-carboxylate

The preparation was accomplished starting from 2.50 g (14.3 mmol) methylindole-5-carboxylate, using glutaric acid monomethyl ester chloride, analogous to the synthesis from step A of example 1.

### B. Production of methyl-3-(4-methoxycarbonylbutanoyl)-1-oxiranylmethylindole-5-carboxylate

The preparation was accomplished starting from 1.54 g (5.08 mmol) methyl-3-(4-methoxycarbonylbutanoyl)indole-5-carboxylate from step A analogous to the synthesis from step C of example 1. The reaction time lasted 1.5 hours. The preparation was purified using column chromatography on silica gel with the eluent ethyl acetate/hexane (step gradient: 1:9-3:7-1:1-7:3), whereby the product accrued as solid material.

### C. Production of methyl-1-[2-hydroxy-3-(4-octylphenoxy)propyl]-3-(4-methoxycarbonylbutanoyl)indole-5-carboxylate

The preparation was accomplished using 603 mg (1.68 mmol) methyl-3-(4-methoxycarbonylbutanoyl)-1-oxiranylmethylindole-5-carboxylate from step B analogous to the synthesis from step D of example 1. Departing therefrom, the preparation was heated for 30 minutes at 100° C. Purification was accomplished using column chromatography on silica gel with the flow medium ethyl acetate/hexane (step gradient: 1:2-1:1). The product was obtained as solid material.

### D. Production of methyl-3-(4-methoxycarbonylbutanoyl)-1-[3-(4-octylphenoxy)-2-oxopropryl]indole-5-carboxylate

440 mg (1.04 mmol) of Dess-Martin periodinane reagent (AlfaAesar) were added in portions to a solution of 514 mg (0.91 mmol) methyl-1-[2-hydroxy-3-(4-octylphenoxy)propyl]-3-(4-methoxycarbonylbutanoyl)indole-5-carboxylate from step C in 5 ml pure methylene dichloride. The resulting suspension was stirred for 6 hours at room temperature. Then the reaction mixture was added into a mixture of aqueous sodium thiosulfate and saturated aqueous sodium hydrogen carbonate solution (1:1). After exhaustive extraction of the aqueous phase with ethyl acetate, drying of the combined organic phases over sodium sulfate, and filtration, the solution was evaporated and the residue was purified using column chromatography on silica gel with the eluent ethyl acetate/hexane (step gradient: 1:2-1:1.5). The product accrued as solid material.

### E. Production of methyl-1-[2,2-diethoxy-3-(4-octylphenoxy)propyl]-3-(4-methoxycarbonylbutanoyl)indole-5-carboxylate

1.3 ml (7.82 mmol) orthoformic acid triethyl ester were added by drops to a solution of 375 mg (0.66 mmol) methyl-3-(4-methoxycarbonylbutanoyl)-1-[3-(4-octylphenoxy)-2-oxopropryl]indole-5-carboxylate from step D in 15 ml pure ethanol. The mixture was mixed with 4 drops of concentrated sulfuric acid and heated for 3 hours to reflux. Then the reaction preparation was introduced into 5% aqueous sodium hydrogen carbonate solution and extracted 3 times with ethyl acetate. The combined organic phases were dried over sodium sulfate, filtered, and the solvent was removed. The raw product was dissolved in toluene and purified by column chromatography on silica gel (ethyl acetate/hexane 2:8). The product was isolated as oil.

### F. Production of 3-(4-carboxybutanoyl)-1-[3-(4-octylphenoxy)-2-oxopropyl]indole-5-carboxylic acid

103 mg (0.16 mmol) of methyl-1-[2,2-diethoxy-3-(4-octylphenoxy)propyl]-3-(4-methoxycarbonylbutanoyl)indole-5-carboxylate from step E were dissolved in heat into 15 ml methanol. After adding thereto a solution of 750 mg (19.5 mmol) sodium hydroxide in 15 ml of water, the resulting solution was stirred for 3 hours while being heated to reflux. Then the methanol was removed by distillation, acidified with 10 ml 4.8 M hydrochloric acid, and extracted 3 times with ethyl acetate. The combined organic phases were dried over sodium sulfate, filtered, and evaporated. The residue was mixed with 15 ml tetrahydrofuran and 3 ml 6 M hydrochloric acid and was again heated under reflux for 1.5 hours. After cooling to room temperature and the addition of 10 ml water threefold extraction of the aqueous phase was accomplished with ethyl acetate. The combined organic phases were dried, filtered, and evaporated. The residue was purified by column chromatography on silica gel (ethyl acetate/hexane/formic acid 3:7:0.5), whereby the product according to formula (2) was obtained as solid material.

### Example 3

### Production of the compound according to the formula (3), 3-(5-carboxypentanoyl)-1-[3-(4-octylphenoxy)-2-oxopropyl]indole-5-carboxylic acid

### A. Production of methyl-3-(5-methoxycarbonylpentanoyl)indole-5-carboxylate

The preparation was accomplished starting from 2.0 g (11 mmol) methylindole-5-carboxylate, using adipic acid monomethyl ester chloride, analogous to the synthesis from step A of example 1.

### B. Production of methyl-3-(5-methoxycarbonylpentanoyl)-1-oxiranylmethyl indole-5-carboxylate

The preparation was accomplished starting from 1.3 g (4.10 mmol) methyl-3-(5-methoxycarbonylpentanoyl)indole-5-carboxylate from step A analogous to the synthesis from step C of example 1. The reaction time lasted 1.5 hours. The preparation was purified using column chromatography on silica gel with ethyl acetate/hexane as eluent (step gradient: 1:9-3:7-1:1), whereby the product accrued as solid material.

### C. Production of methyl-1-[2-hydroxy-3-(4-octylphenoxy)propyl]-3-(5-methoxycarbonylpentanoyl)indole-5-carboxylate

The preparation was accomplished starting from 900 mg (2.41 mmol) methyl-3-(5-methoxycarbonylpentanoyl)-1-oxiranylmethyl indole-5-carboxylate from step B analogous to the synthesis from step D of example 1. Departing therefrom, the preparation was heated for 30 minutes at 100° C. Purification was accomplished using column chromatography on silica gel with ethyl acetate/hexane as eluent (step gradient: 1:2-1:1). The product was obtained as solid material.

### D. Production of methyl-3-(5-methoxycarbonylpentanoyl)-1-[3-(4-octylphenoxy)-2-oxopropyl]indole-5-carboxylate

The preparation was accomplished starting from 751 mg (1.29 mmol) methyl-1-[2-hydroxy-3-(4-octylphenoxy)propyl]-3-(5-methoxycarbonylpentanoyl)indole-5-carboxylate from step C analogous to the synthesis from step D of example 2. Departing therefrom, the reaction time lasted 2 hours.

### E. Production of methyl-1-[2,2-dimethoxy-3-(4-octylphenoxy)propyl]-3-(5-methoxycarbonylpentanoyl)-indole-5-carboxylate

The preparation was accomplished starting from 693 mg (1.20 mmol) methyl-3-(5-methoxycarbonylpentanoyl)-1-[3-(4-octylphenoxy)-2-oxopropyl]indole-5-carboxylate from step D, dissolved into 20 ml pure methanol, and 1.5 ml (13.3 mmol) orthoformic acid trimethyl ester as well as 3 drops of concentrated sulfuric acid analogous to the synthesis from step E of example 2.

### F. Production of 3-(5-carboxypentanoyl)-1-[3-(4-octylphenoxy)-2-oxopropyl]indole-5-carboxylic acid

180 mg (0.29 mmol) methyl-1-[2,2-dimethoxy-3-(4-octylphenoxy)propyl]-3-(5-methoxycarbonylpentanoyl)-indole-5-carboxylate from step E were dissolved in heat into 15 ml methanol. After adding a solution of 1.4 g (35.0 mmol) sodium hydroxide in 15 ml water, the resulting solution was stirred for 2.5 hours while being heated to reflux. Then the methanol was removed by distillation, acidified with 17 ml 6 M hydrochloric acid, and extracted three times with ethyl acetate. The combined organic phases were dried over sodium sulfate, filtered, and evaporated. The residue was mixed with 15 ml tetrahydrofuan and 3 ml 6 M hydrochloric acid and again heated under reflux for 3 hours. After cooling to room temperature and adding 10 ml water, threefold extraction of the aqueous phase was accomplished with ethyl acetate. The combined organic phases were dried, filtered, and evaporated. The residue was purified using column chromatography on silica gel with ethyl acetate/hexane/formic acid as eluent (step gradient: 2:8:0.5-5:5:0.5). The product according to formula (3) was isolated as solid material.

### Example 4

### Production of the compound according to formula (4), 3-(3-carboxypropanoyl)-1-{2-oxo-3-[4-(4-trifluormethylphenoxy)phenoxy]propyl}indole-5-carboxylic acid

### A. Production of methyl-3-(3-methoxycarbonylpropanoyl-1-oxiranylmethylindole-5-carboxylate

The preparation was accomplished starting from 1.30 g (4.49 mmol) methyl-3-(3-methoxycarbonylpropanoyl)indole-5-carboxylate from step A of example 1 analogous to the synthesis from step C from example 1. The reaction time was different, taking 18 hours. Column chromatographic purification on silica gel with ethyl acetate/hexane as eluent (step gradient: 1:9-1:1-8:2) delivered the product as solid material.

### B. Production of methyl-1-{2-hydroxy-3-[4-(4-trifluormethylphenoxy)phenoxy]propyl}-3-(3-methoxycarbonoylpropanoyl)indole-5-carboxylate

The preparation was accomplished starting from 900 mg (2.61 mmol) methyl-3-(3-methoxycarbonyl propanoyl-1-oxiranylmethylindole-5-carboxylate from step A using 660 mg (2.61 mmol) 4-(4-trifluormethyl phenoxy)phenol and 64 mg (0.51 mmol) 4-dimethylaminopyridine analogous to the synthesis from step D of example 1. Departing therefrom, the preparation was heated for 30 minutes at 100° C. Purification was accomplished using column chromatography on silica gel (ethyl acetate/hexane 1:1). The product was isolated as solid material.

### C. Production of methyl-3-(3-methoxycarbonylpropanoyl)-1-{2-oxo-3-[4-(4-trifluormethylphenoxy)phenoxy]propyl}indole-5-carboxylate

The preparation was accomplished starting from 706 mg (1.18 mmol) methyl-1-{2-hydroxy-3-[4-(4-trifluormethylphenoxy)phenoxy]propyl}-3-(3-methoxycarbonoylpropanoyl)indole-5-carboxylate from step B analogous to the synthesis from step D of example 2. Departing therefrom, the reaction time lasted 2 hours.

### D. Production of methyl-1-{2,2-diethoxy-3-[4-(4-trifluormethylphenoxy)phenoxy]propyl}-3-(3-methoxycarbonylpropanoyl)indole-5-carboxylate

The preparation was accomplished starting from 690 mg (1.16 mmol) methyl-3-(3-methoxycarbonylpropanoyl)-1-{2-oxo-3-[4-(4-trifluormethylphenoxy)phenoxy]propyl}indole-5-carboxylate from step C analogous to the synthesis from step E of example 2. Departing therefrom, the reaction time lasted 1.5 hours. After purification by column chromatography on silica gel (ethyl acetate/hexane 2:8) the product was isolated in the form of a solid material.

### E. Production of 3-(3-carboxypropanoyl)-1-{2-oxo-3-(4-(4-trifluormethylphenoxy)-phenoxy)propyl}indole-5-carboxylic acid

289 mg (0.43 mmol) methyl-1-{2,2-diethoxy-3-[4-(4-trifluormethylphenoxy)phenoxy]propyl}-3-(3-methoxycarbonylpropanoyl)indole-5-carboxylate from step D were dissolved in heat in 30 ml methanol. After adding a solution of 2.07 g (52 mmol) sodium hydroxide in 20 ml water, the resulting solution was heated for six hours to reflux while be stirred. After cooling to room temperature, it was acidified with 32 ml of 4.5 M hydrochloric acid. The precipitate was dissolved by adding 15 ml tetrahydrofuran and the reaction mixture was again heated under reflux for 3 hours. The solution was then concentrated to the point that formation of precipitate was observed. After addition of ethyl acetate, the organic phase was separated and the aqueous phase was extracted three more times with ethyl acetate. The combined organic phases were dried, filtered, and evaporated. The residue was purified using column chromatography on silica gel with the flow medium ethyl acetate/hexane/formic acid (step gradient: 2:8:0.1-5:5:0.1-8:2:0.1). The product according to formula (4) was thereby obtained as solid material.

### Example 5

### Production of the compound according to formula (5), 3-(3-methoxycarbonylpropanoyl)-1-{2-oxo-3-[4-(4-trifluormethylphenoxy)phenoxy]propyl}indole-5-carboxylic acid

The compound according to formula (5) was isolated by step E of example 4 as by-product.

### Example 6

### Production of the compound according to formula (6), 1-[3-(4-octylphenoxy)-2-oxopropyl]-3-(2,2,2-trifluoracetyl)indole-5-carboxylic acid

Under nitrogen at 0° C. and while being stirred, a solution of 9.6 ml (67.9 mmol) trifluoroacetic anhydride in 140 ml pure methylene chloride was mixed with 690 mg (1.44 mmol) tert-butyl-1-[3-(4-octylphenoxy)-2-oxopropyl]indole-5-carboxylate, produced according to the production in step C of example 9 from WO 2004/069797. The reaction mixture was stirred for 3 days at room temperature. After evaporation of the solvent, until less than half its volume, the resulting mixture was mixed with hexane until cloudy. The precipitate was drawn off and purified using column chromatography on silica gel (ethyl acetate/hexane/formic acid 1:3:0.1). The product according to formula (6) was obtained as solid material.

### Example 7

### Production of the compound according to formula (7), 3-(3-methyl-1,2,4-oxadiazol-5-yl)-1-[3-(4-octylphenoxy)-2-oxopropyl]indole-5-carboxylic acid

### A. Production of tert-butyl-3-(3-methyl-1,2,4-oxadiazol-5-yl)indole-5-carboxylate

The solution of 135 mg (1.82 mmol) N′-hydroxyacetamidine in 30 ml pure tetrahydrofuran was mixed under nitrogen with 73 mg (1.82 mmol) sodium hydride (60% dispersion in mineral oil) and stirred for 1 hour at room temperature. After adding 500 mg (1.82 mmol) 5-tert-butyl-3-methylindole-3,5-dicarboxylate, produced according to the preparation in step A from example 22 of WO 2004/069797, the preparation was heated under reflux for 24 hours. After cooling to room temperature and the addition of 150 ml water as well as 150 ml ethyl acetate, the mixture was concentrated to remove the tetrahydrofuran. It was then extracted three times with ethyl acetate. The combined organic phases were dried over sodium sulfate, filtered, and evaporated. The residue was purified by column chromatography on silica gel (ethyl acetate/hexane 3:7), whereby the product was obtained as solid material.

### B. Production of tert-butyl-3-(3-methyl-1,2,4-oxadiazol-5-yl)-1-oxiranylmethylindole-5-carboxylate

The preparation was accomplished starting from 305 mg (1.02 mmol) tert-butyl-3-(3-methyl-1,2,4-oxadiazol-5-yl)indole-5-carboxylate from step A analogous to the synthesis from step C of example 1. The preparation was purified using column chromatography on silica gel with flow medium ethyl acetate/hexane (step gradient: 1:9-1:1), whereby the product accrued as solid material.

### C. Production of tert-butyl-1-[2-hydroxy-3-(4-octylphenoxy)propyl]-3-(3-methyl-1,2,4-oxadiazol-5-yl)-indole-5-carboxylate

The preparation was accomplished starting from 150 mg (0.42 mol) tert-butyl-3-(3-methyl-1,2,4-oxadiazol-5-yl)-1-oxiranylmethylindole-5-carboxylate from step B analogous to the synthesis from step D of example 1. Departing therefrom, the preparation was heated for 1 hour at 120° C. Purification was accomplished using column chromatography on silica gel (ethyl acetate/hexane 3:7). The product was obtained as oil.

### D. Production of tert-butyl-3-(3-methyl-1,2,4-oxadiazol-5-yl)-1-[3-(4-octylphenoxy)-2-oxopropyl]indole-5-carboxylate

The preparation was accomplished starting from 135 mg (0.24 mmol) tert-butyl-1-[2-hydroxy-3-(4-octylphenoxy)propyl]-3-(3-methyl-1,2,4-oxadiazol-5-yl)-indole-5-carboxylate from step C analogous to the synthesis from step E of example 1. The product was isolated as oil after purification by column chromatography on silica gel (ethyl acetate/hexane 2:8).

### E. Production of 3-(3-methyl-1,2,4-oxadiazol-5-yl)-1-[3-(4-octylphenoxy)-2-oxopropyl]indole-5-carboxylic acid

A solution of 46 mg (0.08 mmol) tert-butyl-3-(3-methyl-1,2,4-oxadiazol-5-yl)-1-[3-(4-octylphenoxy)-2-oxopropyl]indole-5-carboxylate from step D in 10 ml pure methylene chloride was mixed with 0.5 ml (6.57 mmol) trifluoroacetic acid. After being stirred for 4 hours at room temperature, the mixture was evaporated until dry. Triple co-distillation with hexane delivered the raw product according to formula (7) in form of a solid material that was recrystallized from ethyl acetate.

### Example 8

### Production of the compound according to formula (8), 3-(3-methyl-1,2,4-oxadiazol-5-yl)-1-[2-oxopropyl-3-(4-phenoxyphenol)]indole-5-carboxylic acid

### A. Production of tert-butyl-1-[2-hydroxy-3-(4-phenoxyphenol)propyl]-3-(3-methyl-1,2,4-oxadiazol-5-yl)indole-5-carboxylate

The preparation was accomplished starting from 122 mg (0.34 mmol) tert-butyl-3-(3-methyl-1,2,4-oxadiazol-5-yl)-1-oxiranylmethylindole-5-carboxylate from step B of example 7, using 64 mg (0.34 mmol) 4-phenoxyphenol and 8 mg (0.03 mmol) 4-dimethylaminopyridine, analogous to the synthesis from step D of example 1. Departing therefrom, the preparation was heated for 1 hour at 120° C. Purification was accomplished by column chromatography on silica gel (ethyl acetate/hexane 3:7). The product was isolated as oil.

### B. Production of tert-butyl-3-(3-methyl-1,2,4-oxadiazol-5-yl)-1-[2-oxopropyl-3-(4-phenoxyphenol)]indole-5-carboxylate

The preparation was accomplished starting from 67 mg (0.12 mmol) tert-butyl-1-[2-hydroxy-3-(4-phenoxyphenol)propyl]-3-(3-methyl-1,2,4-oxadiazol-5-yl)indole-5-carboxylate from step A analogous to the synthesis from step E of example 1. The product was obtained as oil after purification by column chromatography on silica gel (ethyl acetate/hexane 2:8).

### C. Production of 3-(3-methyl-1,2,4-oxadiazol-5-yl)-1-[2-oxopropyl-3-(4-phenoxyphenol)]indole-5-carboxylic acid

A solution of 18 mg (0.03 mmol) tert-butyl-3-(3-methyl-1,2,4-oxadiazol-5-yl)-1-[2-oxopropyl-3-(4-phenoxyphenol)]indole-5-carboxylate from step B in 5 ml pure methylene chloride was mixed with 0.2 ml (2.6 mmol) trifluoroacetic acid. After being stirred for 6 hours at room temperature, the mixture was evaporated until dry. Triple co-distillation with hexane delivered the raw product in the form of a solid material. This solid material was dissolved in acetonitrile. After addition of water, the acetonitrile was distilled off and then the water was removed by freeze-drying, whereby the product according to formula (8) remained as solid material.

### Example 9

### Production of the compound according to formula (12), 3-(5-carboxypentanoyl)-1-[3-(4-phenylphenoxy)-2-oxopropyl]indole-5-carboxylic acid

### A. Production of methyl-3-(5-methoxycarbonylpentanoyl)indole-5-carboxylate

A suspension of 1.80 g (13.5 mmol) AlCl₃ in 15 ml dry CH₂Cl₂ was mixed with 1.0 ml (5.9 mmol) hexanedioic acid monomethyl ester chloride. After 1 hour of stirring at room temperature, 700 mg (4.0 mmol) methylindole-5-carboxylate were added. After another 30 minutes of stirring at room temperature, the reaction preparation was poured into water and extracted with a mixture of ethyl acetate and CH₂Cl₂. The organic phase was initially washed with 5% aqueous Na₂CO₃ solution and then with water. After drying over Na₂SO₄, it was evaporated, whereby the product was precipitated as solid material.

### B. Production of methyl-3-(5-methoxycarbonylpentanoyl)-1-oxiranylmethylindole-5-carboxylate

The preparation was accomplished starting from methyl-3-(5-methoxycarbonylpentanoyl)indole-5-carboxylate from step A, corresponding to the synthesis of step B as described in example 3.

### C. Production of methyl-1-[2-hydroxy-3-(4-phenylphenoxy)propyl]-3-(5-methoxycarbonylpentanoyl)indole-5-carboxylate

187 mg (0.5 mmol) methyl-3-(5-methoxycarbonylpentanoyl)-1-oxiranylmethylindole-5-carboxylate from step B, 85 mg (0.5 mmol) 4-phenylphenol, and 12 mg 4-dimethylaminopyridine were dissolved in a little CH₂Cl₂. Then the solvent was distilled off and the residue was heated for 75 minutes under nitrogen in an oil bath at 110° C. After cooling, the preparation was dissolved in a little CHCl₃. Purification was accomplished by column chromatography on silica gel with petroleum ether/ethyl acetate (1:1) as eluent. The product was obtained as a wax-like substance.

### D. Production of methyl-3-(5-methoxycarbonylpentanoyl)-1-[2-oxo-3-(4-phenylphenoxy)propyl]-indole-5-carboxylate

208 mg (0.49 mmol) Dess-Martin periodinane reagent (AlfaAesar) were added under nitrogen to a solution of 140 mg (0.26 mmol) methyl-1-[2-hydroxy-3-(4-phenylphenoxy)-propyl]-3-(5-methoxycarbonylpentanoyl)indole-5-carboxylate from step C in 4 ml pure methylene chloride. The mixture was stirred for 2 hours at room temperature. After addition of a solution of 0.5 g sodium sulfate in 10 ml saturated aqueous sodium hydrogen carbonate solution, it was extracted with ethyl acetate. The organic phase was washed with saturated aqueous sodium chloride solution and dried over sodium sulfate. After distilling off the solvent, the residue was purified using column chromatography on silica gel with petroleum ether/ethyl acetate (1:1) as eluent. The product accrued as solid material.

### E. Production of 3-(5-carboxypentanoyl)-1-[2-oxo-3-(4-phenylphenoxy)propyl]indole-5-carboxylic acid

A mixture of 110 mg (0.20 mmol) methyl-3-(5-methoxycarbonylpentanoyl)-1-[2-oxo-3-(4-phenylphenoxy)propyl]-indole-5-carboxylate from step D, 30 ml ethanol, and 10 ml aqueous 10% KOH was stirred for 15 hours under nitrogen at room temperature. After adding water, it was acidified with HCl and extracted with ethyl acetate. The organic phase was washed with diluted HCl, dried, and the solvent was distilled off. The residue was purified using column chromatography on silica gel (petroleum ether/ethyl acetate/formic acid 4:6:0.1). The product fractions were evaporated to a few ml and the product according to the formula (12) precipitated by adding hexane.

### Example 10

Production of the compound according to formula (13), 3-(5-carboxypentanoyl)-1-[3-(4-phenoxyphenoxy)-2-oxopropyl]indole-5-carboxylic acid

### A. Production of methyl-1-[2-hydroxy-3-(4-phenoxyphenoxy)propyl]-3-(5-methoxycarbonylpentanoyl)indole-5-carboxylate

The preparation was accomplished starting from 187 mg (0.50 mmol) methyl-3-(5-methoxycarbonylpentanoyl)-1-oxiranylmethylindole-5-carboxylate from step B of example 9 analogous to the synthesis from step C of example 9 using 93 mg (0.50 mmol) 4-phenoxyphenol and 10 mg 4-dimethylaminopyridine. Departing therefrom, the preparation was heated for 90 minutes at 110° C. Purification was accomplished by column chromatography on silica gel (petroleum ether/ethyl acetate 6:4). The product according to formula (13) was obtained as solid material.

### B. Production of methyl-3-(5-methoxycarbonylpentanoyl)-1-[2-oxo-3-(4-phenoxyphenoxy)propyl]indole-5-carboxylate

The preparation was accomplished starting from 100 mg (0.18 mmol) methyl-1-[2-hydroxy-3-(4-phenoxyphenoxy)propyl]-3-(5-methoxycarbonylpentanoyl)indole-5-carboxylate from step A analogous to the synthesis from step D as stated in example 9.

### C. Production of 3-(5-carboxypentanoyl)-1-[2-oxo-3-(4-phenoxyphenoxy)propyl]indole-5-carboxylic acid

The preparation was accomplished starting from 70 mg (0.13 mmol) methyl-3-(5-methoxycarbonylpentanoyl)-1-[2-oxo-3-(4-phenoxyphenoxy)propyl]indole-5-carboxylate from step B analogous to the synthesis from step E as stated in example 9. The ethyl acetate extract was evaporated to a few ml and the product according to formula (13) precipitated by adding petroleum ether.

### Example 11

Production of the compound according to formula (14), 3-(4-carboxybenzoyl)-1-[3-(4-octylphenoxy)-2-oxopropyl]indole-5-carboxylic acid

### A. Production of methyl-3-(4-methoxycarbonylbenzoyl)indole-5-carboxylate

A suspension of 1.08 g (8.1 mmol) AlCl₃ in 10 ml dry CH₂Cl₂ was mixed with 0.50 g (2.5 mmol) terepthalic acid monomethyl ester chloride. After 5 minutes of stirring at room temperature, 0.46 g (2.6 mmol) methylindole-5-carboxylate were added thereto. After another 4 hours of stirring at room temperature, a mixture of water and tetrahydrofuran (THF) (1:1) was added to the reaction preparation, which was then twice extracted with CH₂Cl₂. The combined organic phases were initially washed with 5% aqueous Na₂CO₃ solution and then with water. After drying over Na₂SO₄, it was evaporated to a few ml. After the addition of ethyl acetate and further evaporation, the product precipitated as solid material.

### B. Production of methyl-3-(4-methoxycarbonylbenzoyl)-1-oxiranylmethylindole-5-carboxylate

240 mg (0.71 mmol) methyl-3-(4-methoxycarbonylbenzoyl)indole-5-carboxylate from step A were mixed with 81 mg (1.27 mmol) powdered 88% potassium hydroxide and 22 mg (0.07 mmol) tetrabutylammonium bromide. After adding 2.0 ml (26 mmol) epichlorohydrin, it was stirred for 75 minutes at room temperature. Then the preparation was applied directly onto a silica gel column and eluted with petroleum ether/ethyl acetate (step gradient: 9:1-1:2). The eluates were evaporated and the product recrystallized from ethyl acetate/petroleum ether.

### C. Production of methyl-1-[2-hydroxy-3-(4-octylphenoxy)propyl]-3-(4-methoxycarbonylbenzoyl)indole-5-carboxylate

The preparation was accomplished starting from 145 mg (0.37 mmol) methyl-3-(4-methoxycarbonylbenzoyl)-1-oxiranylmethylindole-5-carboxylate from step B analogous to the synthesis from step C as stated in example 9 using 76 mg (0.37 mmol) 4-octylphenol and 9 mg 4-dimethylaminopyridine. Purification was accomplished by column chromatography on silica gel (petroleum ether/ethyl acetate 7:3). The product was obtained as solid material.

### D. Production of methyl-3-(4-methoxycarbonylbenzoyl)-1-[3-(4-octylphenoxy)-2-oxopropyl]indole-5-carboxylate

The preparation was accomplished starting from 95 mg (0.16 mmol) methyl-1-[2-hydroxy-3-(4-octylphenoxy)propyl]-3-(4-methoxycarbonylbenzoyl)indole-5-carboxylate from step C analogous to the synthesis from step D as stated in example 9. Purification was accomplished by column chromatography on silica gel (petroleum ether/ethyl acetate 6:4). The product accrued as a resin-like substance.

### E. Production of 3-(4-carboxybenzoyl)-1-[3-(4-octylphenoxy)-2-oxopropyl]indole-5-carboxylic acid

A mixture of 37 mg (0.062 mmol) methyl-3-(4-methoxycarbonylbenzoyl)-1-[3-(4-octylphenoxy)-2-oxopropyl]indole-5-carboxylate from step D, 15 ml ethanol, and 5 ml aqueous 10% KOH was stirred for 4 hours under nitrogen at room temperature. After the addition of water, it was acidified with HCl and extracted with ethyl acetate. The organic phase was washed with diluted HCl, dried, and the solvent was distilled off. The residue was purified by column chromatography on silica gel, initially with petroleum ether/ethyl acetate/formic acid 6:4:0.1, and then with tetrahydrofuran (THF). After removal of the solvent, the product 3-(4-carboxybenzoyl)-1-[3-(4-octylphenoxy)-2-oxopropyl]indole-5-carboxylic acid according to formula (14) was obtained as solid material.

### Example 12

### Determining Water Solubility

The determination of the water solubility of the compound was accomplished, if not hereinafter stated otherwise, with reference to the method published by Kim et al., J. Med. Chem. 2005, 48, 3621-3629.

1 mg of each of the compounds produced according to examples 1 to 8 according to formulas (1) to (8) were mixed with 2 ml phosphate buffered saline solution (PBS buffer, ph=7.4, 0.01 M KH₂PO₄/K₂HPO₄, 0.0027 M KCl, 0.137 M NaCl at 25° C.), produced by dissolving a phosphate buffered saline tablet, Sigma (catalog number: P4417) in 200 ml deionized water. The mixture was placed in an ultrasonic bath (Sonorex TK52, Bandelin) for 10 minutes and then shaken lightly in a shaking water bath (GFL 1083). Subsequently, the mixture was centrifuged for 10 minutes at 4000×g and room temperature. About 1 ml clear solution was taken from the supernatant. 200 μl of the clear solution were mixed with 250 μl acetonitrile (VWR) and 50 μl 0.1 M phosphoric acid. From this solution, a volume of between 5 μl and 100 μl was injected into an HPLC system (Waters, Waters 717plus Autos ampler, Waters 515 pump, and Waters 2487 UV detector)

The content of dissolved compound was determined using a standard straight line, which was built by injecting different amounts ranging from 5 μl to 100 μl of reference solutions 1 and 2. For reference solution 1, 2 μl of a 5 mM solution of the respective compound in dimethyl sulfoxide (DMSO) were mixed with 198 μl PBS buffer, 250 μl acetonitrile, and 50 μl 0.1 M phosphoric acid. For reference solution 2, 2 μl of a 5 mM solution of the respective compound in DMSO were mixed with 398 μl PBS buffer, 500 μl acetonitrile, and 100 μl 0.1 M phosphoric acid.

C₁₈ Aqua®-columns from the company Phenomenex (Aqua®, RP18, 75×4.6 mm, 3 μm) were used as the stationary phase. The detection wave length was 240 nm; the flow rate was 0.7 ml/min For the compounds from examples 1, 2, 3, and 7 according to the formulas (1), (2), (3), and (7), a mixture of acetonitrile/water/phosphoric acid (85%) in ratios 700:300:1 (v/v/v) was used as the mobile phase, and for the compounds from examples 4, 5, and 8 according to the formulas (4), (5), and (8), a mixture of acetonitrile/water/phosphoric acid (85%) in ratios 530:470:1 (v/v/v) was used, and for the compound from example 6 according to the formula (6), a mixture of acetonitrile/water/phosphoric acid (85%) in ratios 800:200:1 (v/v/v) was used.

In comparative experiments, the water solubility of compounds according to the publication WO 2004/069797 corresponding to the following formulas (9), (10), and (11)

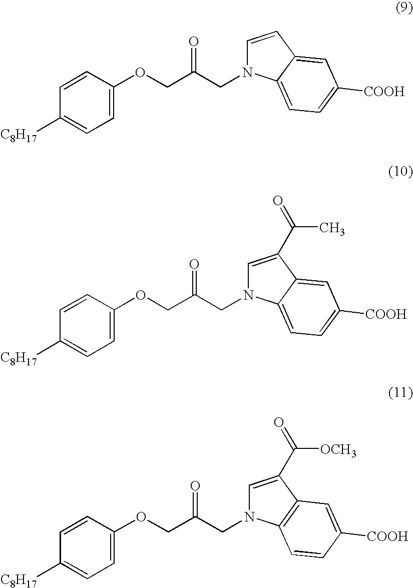

were determined under corresponding conditions.

It could be established that the inventive compounds from examples 1, 2, 3, and 4 according to formulas (1), (2), (3), and (4) exhibited water solubilities between 190 μg/ml and 410 μg/ml. The compounds according to formulas (5) and (8) exhibited water solubilities between 15 μg/ml and 35 μg/ml.

In contrast, the compounds according to formulas (9), (10), and (11) exhibited water solubilities lower than 1 μg/ml.

The inventive compounds especially from examples 1 to 5 corresponding to formulas (1), (2), (3), (4), (5), and (8) thus exhibited improved water solubility, whereby the compounds from examples 1 to 4 in particular, corresponding to formulas (1) to (4), feature considerably increased water solubility.

### Example 13

### Determining the Inhibition of the Cytosolic Phospholipase A₂

The effectiveness of the inventive compounds was determined based on the inhibition of cytosolic phospholipase A₂. The determination was accomplished, if not described otherwise hereinafter, as was described in Schmitt, M.; Lehr, M., “HPLC assay with UV spectrometric detection for the evaluation of inhibitors of cytosolic phospholipase A₂” J. Pharm. Biomed. Anal. 2004, 35, 135-142.

Cytosolic phospholipase A₂ that had been isolated from human thrombocytes was used as the enzyme source. The inhibition of the enzyme activity was ascertained by measurement of the arachidonic acid released by the cleavage of 1-stearoyl-2-arachidonoyl-sn-glycero-3-phosphocholine in the presence or absence of the respective compound being studied.

To produce a solution of covesicles from the substrate 1-stearoyl-2-arachidonoyl-sn-glycero-3-phosphocholine (SAPC) (Sigma) and 1,2-dioleoyl-sn-glycerol (DOG) (Sigma), corresponding amounts of the chloroform solution from SAPC (10 mg/ml) and DOG (5 mg/ml) were mixed and then the chloroform was steamed away in a stream of nitrogen. The residue was mixed with enough Tris buffer (50 ml Tris, 1 mM dithiothreitol, 150 mM NaCl, 1 mM CaCl₂, ph 8 at 20° C.) that a concentration for SAPC of 0.26 mM and a concentration for DOG of 0.13 mM were present. The mixture was homogenized for 10 minutes in an ultrasonic bath at 35° C. for the purpose of forming covesicles.

2 μl of the solution of the respective compound in DMSO (made from a 5 mM stock solution in DMSO) or, in the case of the control value, 2 μl of DMSO, were each mixed with 78 μl of the substrate mixture in Eppendorf tubes.

After 10 minutes of preincubation in a water bath at 37° C., 20 μl of the solution of cytosolic phospholipase A₂ obtained from human thrombocytes were added to each of the 80-ml solutions and this mixture was incubated for another 60 minutes at 37° C. The incubation preparations contained 0.20 mM SAPC and 0.10 mM DOG per 100 μl. After that, the enzyme reaction was stopped by adding 400 μl of a solution of acetronitrile/methanol/0.1 M aqueous EDTA-solution in ratios 16:15:1 (v/v/v), whereby this solution contained 3 μg/ml nordihydroguaiaretic acid (NDGA) (Sigma) as antioxidant and 1.55 μg/ml 4-undecyloxybenzoic acid as internal standard. Subsequently, the samples were placed in ice for 10 to 15 minutes and then stored at −20° C. until solid phase extraction.

The octadecyl solid phase extraction columns with a bed volume of 200 mg and a capacity of 3 ml (Baker) were initially washed with 6 ml methanol and then with 6 ml water. The samples were diluted with 2 ml 0.005 M aqueous NaOH and then introduced to the solid phase columns. After washing with 1 ml water, the bound arachidonic acids eluted with 3×200 μl methanol. The eluate was mixed with 600 μl water. 100 μl of this solution was injected into the HPLC apparatus (Waters, Waters 717plus Autosampler, Waters 515 pump, and Waters 2487 UV detector). Data analysis was accomplished using the software program Millennium. For the column, a Nucleosil 100-3 C18 column (125×3 mm) with a Nucleosil 100-3 C18 pre-column (20×3 mm) (CS-Chromatographie-Service, Langerwehe) was used. The flow rate was 0.4 ml/min; the detection wavelength was 200 nm. A mixture of acetonitrile/water/phosphoric acid (85%) in ratios 770:230:1 (v/v/v) was used for the flow medium. The chromatogram run time was 30 minutes. Before the next injection, the column was always equilibrated for 15 minutes.

It was found that at a concentration of 0.1 μM, the inventive compounds from examples 1, 2, 4, 5, 6, and 7 corresponding to formulas (1), (2), (4), (5), (6), and (7) inhibited the activity of the cytosolic phospholipase A₂ from 30% to 97% compared to the control value, for which, instead of the solution of the compound in DMSO, pure DMSO was substituted.

Furthermore, the IC₅₀ value for inhibiting cytosolic phospholipase A₂ using the compounds from examples 1 to 8 corresponding to the formulas (1) to (8) was ascertained.

The IC₅₀ values were calculated from the values of cytosolic phospholipase A₂ inhibition obtained from different concentrations with the help of the Probit model (see Hartke, Mutschler, DAB 9 Kommentar Band 1 S. 733-734, Wissenschaftliche Verlagsgesellschaft Stuttgart 1978).

The IC₅₀ value of the compounds for the inhibition of cytosolic phospholipase A₂ corresponds to the concentration of the compound that is necessary to reduce the activity of the enzyme by 50%. The lower the IC₅₀ value, the more the compound inhibits cytosolic phospholipase A₂.

So the compound from example 1 according to formula (1) exhibited an IC₅₀ value of 0.21 μM; the compound from example 2 according to formula (2) exhibited an IC₅₀ value of 0.03 μM; the compound from example 3 according to formula (3) exhibited an IC₅₀ value of 0.022 μM; the compound from example 4 according to formula (4) exhibited an IC₅₀ value of 0.19 μM; and the compound from example 5 according to formula (5) exhibited an IC₅₀ value of 0.022 μM.

The compound from example 6 according to formula (6) exhibited an IC₅₀ value of 0.007 μM; the compound from example 7 according to formula (7) exhibited an IC₅₀ value of 0.002 μM; and the compound from example 8 according to formula (8) exhibited an IC₅₀ value of 0.007 μm.

This shows that the inventive compounds are effective at inhibiting cytosolic phospholipase A₂, whereby the effectiveness of the compounds from examples 6, 7, and 8 corresponding to formulas (6), (7), and (8) is better than the effectiveness of the compounds from examples 1 to 5 corresponding to formulas (1) to (5).

In particular, the compounds from examples 1 to 5 and 8 corresponding to formulas (1) to (5) and (8), especially the compounds from examples 1 to 4 corresponding to formulas (1) to 4, were able to exhibit good solubility as well as good inhibition of cytosolic phospholipase A₂ activity.

### Example 14

### Determining the anti-inflammatory properties of the compound according to formula (3), 3-(5-carboxypentanoly)-1-[3-(4-octylphenoxy)-2-oxopropyl]indole-5-carboxylic acid

The determination of the anti-inflammatory properties in vivo in the model of contact dermatitis induced by benzalkonium chloride was accomplished, if not stated otherwise hereinafter, according to the method published by E. Hyun et al., British Journal of Pharmacology, 2004, 143, S. 618-625.

BALB/c mice (Harlan Winkelmann GmbH, Borchen) were used as laboratory animals. A contact dermatitis was induced by introducing 10 μl per ear of a 5% benzalkonium chloride solution (Sigma) in olive oil/acetone (1:5) to the dorsal side of both ears for each of 8 lab animals per experimental group. This led to a swelling of the ears.

After 10 minutes, 10 μl acetone were applied onto the dorsal sides of both ears of each animal in a negative control group of 8 untreated animals; 10 μl of a 1% solution (m/V) of the compound according to formula (3) in acetone corresponding to 0.1 mg/ear were applied to an experimental group of 8 animals; and 10 μl of a 0.05% clobetasol-17-propionate solution (Karison Crinale, Dermapharm AG) corresponding to 0.05 mg/ear were applied to a positive control group of 8 animals. After 1 hour, 3 hours, 5 hours, 7 hours, 24 hours, 48 hours, and 72 hours, the ear thicknesses were measured using a digital caliper (Roth, Karlsruhe).

It was found that the application of the inventive compound according to formula (3) in the experimental group led to a significantly decreased increase in ear thickness compared to the negative control untreated animals. This shows that the compound according to formula (3) 3-(5-carboxypentanoly)-1-[3-(4-octylphenoxy)-2-oxopropyl]indole-5-carboxylic acid exhibits an anti-inflammatory effect.

1. Compounds of the general formula (I), as given below 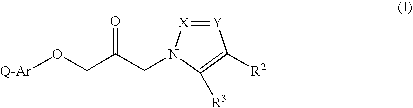
wherein:
Q represents R¹, OR¹, SR¹, SOR¹, SO₂R¹, NR⁹R¹ or a straight-chained C₁₋₃₁ alkyl or C₂₋₃₁ alkenyl or alkynyl residue, which may be interrupted by 1 or 2 residues, independently chosen from O, S, SO, SO₂, NR⁹, and aryl, which can be substituted with 1 or 2 substituents R⁴, and which can be substituted with 1 to 4 C₁₋₆ alkyl residues and/or 1 or 2 aryl residues, whereby the aryl residues can be substituted with 1 or 2 substituents R⁴;
Ar represents an aryl residue, which can be substituted with 1 or 2 substituents R⁴;
X represents N or CR⁵;
R¹ represents H or an aryl residue, which can be substituted with 1 or 2 substituents R⁴;
R² and R³
a) Independently represent H, C₁₋₆ alkyl, C₂₋₆ alkenyl, C₂₋₆ alkynyl, or R⁷—W, or
b) together with the carbon atoms to which they are bound, represent a 5- or 6-membered aromatic or heteroaromatic ring, which can be substituted with 1 or 2 substituents R⁴;
R⁴ represents C₁₋₆ alkyl, halogen, CF₃, CN, NO₂, OR⁹, S(O)_{O}R⁹, COR⁹, COOR⁹, CONR⁹R¹⁰, SO₃R⁹, SO₂NR⁹R¹⁰, tetrazolyl or R⁷—W;
R⁵ represents H or R⁴;
R⁷ represents C₁₋₆ alkyl, C₂₋₆ alkenyl, or C₂₋₆ alkynyl;
R⁹ represents H, C₁₋₆ alkyl, or aryl;
R¹⁰ represents H or C₂₋₆ alkyl;
W represents COOH, SO₃H, or tetrazolyl; and
o represents 0, 1, or 2;
and/or their enantiomers, diastereomers, as well as their pharmaceutically acceptable salts and/or esters, wherein
Y represents CR¹²,
wherein
R¹² is chosen from the group comprising 3-methyl-1,2,4-oxadiazol-5-yl and/or COR¹³,
R¹³ is chosen from the group comprising CF₃, E and/or D-E;
E is chosen from the group comprising COOH, COOR¹⁴, CONR¹⁴R¹⁵, SO₃R¹⁴, and/or SO₂NR¹⁴R¹⁵;
D is chosen from the group comprising C₁₋₁₀ alkyl, C₂₋₁₀ alkenyl, or C₂₋₁₀ alkynyl, aryl, T-aryl, T-aryl-G and/or aryl-G;
T,G are chosen identically or independently of each other from the group comprising C₁₋₁₀ alkyl, C₂₋₁₀ alkenyl, and/or C₂₋₁₀ alkynyl;
R¹⁴, R¹⁵ are chosen identically or independently from the group comprising H, C₁₋₆ alkyl, and/or aryl.

2. Compounds according to claim 1, wherein
R¹² represents CO—(CH₂)ᵣ—COOR¹⁴,
wherein
r is 1, 2, 3, 4, or 5, preferably 2, 3, or 4.

3. Compounds according to claim 1 wherein
Q represents C₅-C₁₂ alkyl, preferably C₇-C₁₀ alkyl.

4. Compounds according to claim 1 wherein
Q represents OR¹, wherein R¹ represents an aryl residue, which can be substituted with a substituent R⁴, whereby R⁴ preferably represents CF₃.

5. Compound according to claim 1 wherein the compound exhibits the following formula (1):

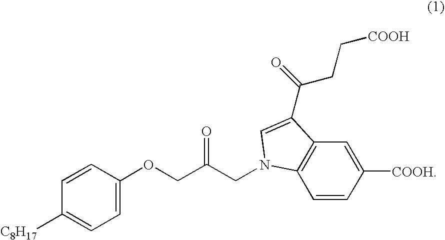

6. Compound according to claim 1 wherein the compound exhibits the following formula (2):

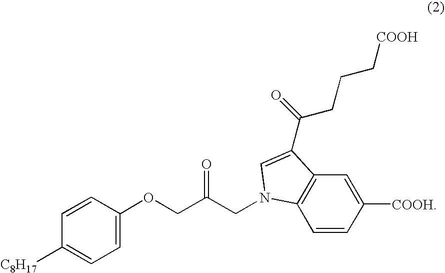

7. Compound according to claim 1 wherein the compound exhibits the following formula (3):

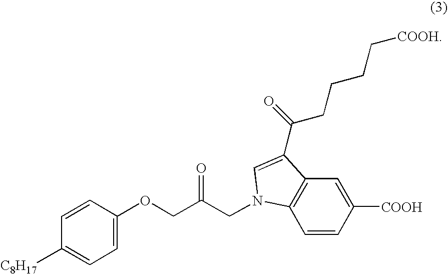

8. Compound according to claim 1 wherein the compound exhibits the following formula (4):

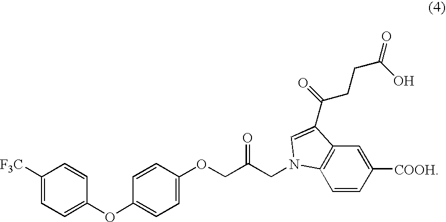

9. Compound according to claim 1 wherein the compound exhibits the following formula (5):

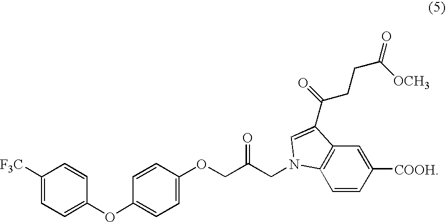

10. Pharmaceutical agent comprising a compound of the general formula (I) according to claim 1 and/or their enantiomers, diastereomers, as well as their pharmaceutically acceptable salts or esters.

11. Use of a compound of the general formula (I) according to claim 1 and/or their enantiomers, diastereomers, as well as their pharmaceutically acceptable salts and/or esters for the production of a pharmaceutical agent for prophylactic and/or therapeutic treatment of illnesses that are caused by or contributed to by an increased activity of phospholipase A₂.

12. Use according to claim 11, wherein the illness is chosen from the group comprising inflammations, pain, fever, allergies, asthma, psoriasis, cerebral ischemia, Alzheimer's disease, chronic skin diseases, damage to the skin by UV rays, rheumatic illnesses, thrombosis, anaphylactic shock, urticaria, acute and chronic rashes and/or endotoxic shock.

13. Method for producing a compound according to the general formula (I) according to claim 1 wherein the compound is according to the following general formula (IV) 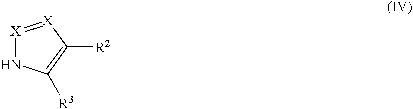
with epichlorohydrin is converted to a compound according to the following general formula (VI) 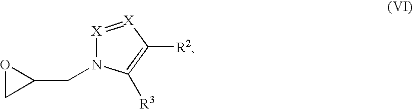
and, further, that the compound of the formula (VI) with a compound according to the following general formula (VII)
Q-Ar—OH (VII)
is converted to a compound according to the following general formula (VIII) 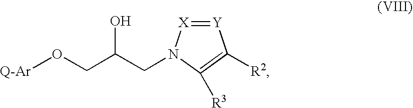
and that the compound (VIII) is oxidized to ketone.
